(19) United States
(12) Patent Application Publication
(10) Pub. No.: US 2019/0355445 A1
(43) Pub. Date: Nov. 21, 2019

(54) Methods and Systems for Identifying Subjects for Enrollment in Clinical Trials

(71) Applicant: Laboratory Corporation of America Holdings, Burlington, NC (US)

(72) Inventor: Steve Jones, Maidenhead (GB)

(21) Appl. No.: 16/411,846

(22) Filed: May 14, 2019

Related U.S. Application Data
(60) Provisional application No. 62/671,202, filed on May 14, 2018.

(51) Int. Cl. G16H 10/20; G16H 10/60

(52) U.S. Cl. CPC G16H 10/20; G16H 10/40; G16H 10/60

(57) ABSTRACT

Embodiments of the present invention are advantageous for facilitating the planning and conduct of clinical trials. In an embodiment, a method is disclosed for facilitating the enrollment phase of clinical trials, and clinical trial planning, by identifying potential subjects and sites for enrollments.

### RELATED APPLICATION

Pursuant to 35 U.S.C. §119, this application claims priority to and filing benefit of U.S. Provisional Application No. 62/671,202 filed May 14, 2018, which is incorporated herein by reference in its entirety.

### FIELD

Disclosed herein are methods and systems for facilitating the planning and conduct of clinical trials. In an embodiment, a method is disclosed for facilitating the enrollment phase of clinical trials, and clinical trial planning, by identifying potential subjects and sites for enrollments.

### BACKGROUND

Clinical trials are an important part of the process of the introduction of new treatments into a healthcare system. Such new treatments may include novel vaccines, compositions (e.g. pharmaceutical compositions), dietary supplements, medical and/or dietary choices, and/or medical devices into a health care system. Clinical trials may be utilized to generate data on safety, efficacy, patient compliance, ease of use and other topics relating to the treatment. Clinical trials may vary in size and costs, and they can involve a single research center or a plurality of research centers in a single country or a plurality of countries.

Organizations that conduct clinical trials generally find investigators who enroll volunteer subjects (e.g. patients) as subjects for the new treatment or treatment/device/procedure to be tested. The clinical trial may require that these subjects undergo lab work at a laboratory or phlebotomy center at the investigator's location. The creation and efficacy of the clinical trial may be reduced if the distance between a subject's location and the location of the center conducting the examinations and tests in accordance with the clinical trial protocol, creates a hindrance to the subject being able to provide necessary samples for lab work.

Accordingly, it would be desirable to have methods and systems for identifying subjects for enrollment in clinical trials that are located sufficiently proximate to a laboratory or a phlebotomy center so that the ability of the subjects to provide samples for laboratory analysis is not hindered, with perhaps, where possible, other examinations and consultations being performed in non-emergency situations, via a telephone conversation with/without video link. This and other advantages are achieved by the methods and systems of the present invention.

### SUMMARY

In an embodiment, the present invention provides a method for identifying subjects for enrollment in a clinical trial comprising:
a) identifying a number of potential investigators for a clinical trial to create an investigator list;
b) determining the location of each investigator;
c) identifying a number of possible subjects for the clinical trial;
d) determining the location of each subject or the location of their current health care pro
e) selecting a specified distance between a subject and an investigator; and
applying a spatial cluster analysis to determine the number of subjects within the specified distance for each investigator.

In an embodiment, a method further comprises: identifying clusters of subjects of a predetermined size outside the specified distance for an investigator; and identifying additional potential investigators within a specified distance of the cluster.

In an embodiment, identifying investigators may be based on criteria including, but not limited to: disease specialty; performance in past clinical trials; performance with respect to enrollment in past clinical trials; location; country; incidence or prevalence of disease in the area; prescription practices in the area; prescription trends in the area and similar criteria.

In an embodiment, identifying subjects and/or determining the location of a subject may be based on deindentified information.

In an embodiment, a spatial cluster analysis creates clusters with a characteristic of interest. A characteristic of interest may comprise one or more of the following: number of possible subjects within a specified distance of an investigator; number of possible subjects within a specified distance of each other; incidence or prevalence of disease in the area; prescription practices in the area; prescription trends in the area and similar criteria.

In an embodiment, the potential investigators are ranked according to the number of subjects within the specified distance and potentially selected /rejected based on this number. In an embodiment, the number of possible subjects within a specified distance of a selected investigator comprises a cluster also referred to as subject referrals for the investigator for the trial. As noted, a spatial cluster analysis may create dusters of subjects within a specified distance of each other but outside the specified distance of the a priori list of identified investigators. In an embodiment, additional investigators identified from other sources of information that meet/exceed the criteria used for the initial list that are within the required distance of such a cluster may be added to the investigator list,

In an embodiment, the method further comprises selecting a specified distance and using the specified distance as a criteria for selecting investigators without use of the initial investigator list.

In another embodiment the present invention provides a method for identifying countries and locations within the countries of subjects for enrollment in a clinical trial, the method comprising:
a) creating a subject profile for a clinical trial;
b) identifying a plurality of subjects based on the subject profile
c) determining the location of each subject or their healthcare provider;
d) conducting a spatial cluster analysis to create at least one cluster;
e) determining the number of subjects and their locations within each cluster;
f) identifying each cluster within a specified distance of an investigator for the clinical trial;
g) determining an optimal number of dusters for the clinical trial.

In an embodiment, creating a subject profile may comprise defining the “ideal” subject profile for the trial that meets the inclusion exclusion criteria and the conditions that would promote 100% adherence to the trial in terms of available time (eg. Overnight hospital stays are least disruptive for individuals with minimal family commitments).

In an embodiment an optimal number of clusters may comprise one or more of the following characteristics: capability to meet the number of subjects needed for the clinical trial; number of investigator sites; number of countries; capability to meet regulator needs; needs of the clinical trial sponsor. For example, the optimal number of clusters may be the number that provides the capability to meet the clinical trial's protocol needs in terms of numbers of subjects in as few countries/investigator sites as possible whilst addressing the regulatory needs of the clinical trial sponsor.

In an embodiment, each possible subject comprises I_{cn(k)} wherein c=1 and n=1 identifies the first investigator location within the first country with k possible subjects within the defined distance (k dependent on c and n); c=1 and n=2 identifies the second possible investigator location within the first country and so on for C countries and n=1, . . . N(c) investigator locations with k(nc) possible subjects.

In an embodiment of the invention, a spatial cluster analysis may be represented by the following formula:

Minimization of number of countries C and Σn_{c}, such that

$$Probability ( {\sum }_{c = 1}^{C} {\sum }_{n = 1}^{N ( c )} {k}_{nc} = Total Required Subjects ) > p$$

Where p is set at a level commensurate with the level of acceptable certainty of meeting study timelines and actual conversion of subjects into clinical trial patients.

In an embodiment, location information may comprise latitude and longitude. GPS data, a zip code, a physical address and/or a postal code may be used as to determine latitude and longitude.

Possible subjects for a clinical trial may comprise one or more of the following attributes:

a disease or condition of interest

a genetic trait of interest

an age or age range of interest

a sex of interest

a health history of interest

a medication or previous treatment regimen of interest

a behavior pattern of interest; and/or

another individual attribute of interest.

Possible investigators for a clinical trial may comprise one or more of the following attributes:

a specific expertise;

a specific experience with past clinical trials;

a specific research experience;

a specific position within the medical profession and/or scientific discovery within the realms of the therapeutic area to such a degree that the individual is recognized as a Key Opinion Leader;

an association with a research institution and/or medical facility;

an ability to interact with patients; and/or

another attribute of interest.

### BRIEF DESCRIPTION OF THE FIGURES

FIG. 1 is a schematic diagram of an embodiment of the present invention.

FIG. 2 is a graphic illustration of potential subjects for enrollment in a clinical trial in an embodiment of the present invention discussed in the Example.

FIG. 3 depicts the location of potential subjects for enrollment in a clinical trial in an embodiment of the present invention discussed in the Example.

FIG. 4 depicts the location of potential investigator sites for enrollment in a clinical trial in an embodiment of the present invention discussed in the Example.

FIG. 5 depicts the location of potential additional investigator sites for enrollment in a clinical trial in an embodiment of the present invention discussed in the Example.

FIG. 6 depicts the location of investigator sites from past clinical trials for enrollment in a clinical trial in an embodiment of the present invention discussed in the Example.

FIG. 7 is a diagrammatic representation of a spatial cluster analysis in an embodiment of the present invention discussed in the Example.

FIG. 8 depicts the location of potential sites for a clinical trial in an embodiment of the present invention discussed in the Example.

### DESCRIPTION

In the following description, various possible embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

It is further noted that, as used in this specification, the singular forms “a,” “an,” and “the” include plural referents unless expressly and unequivocally limited to one referent. The term “and/or” generally is used to refer to at least one or the other. In some case the term “and/or” is used interchangeably with the term “or.” The term “including” is used herein to mean, and is used interchangeably with, the phrase “including but not limited to.” The term “such as” is used herein to mean, and is used interchangeably with, the phrase “such as but not limited to.”

Unless defined otherwise, all technical and scientific terms used herein have the same meaning as commonly understood by one of ordinary skill in the art.

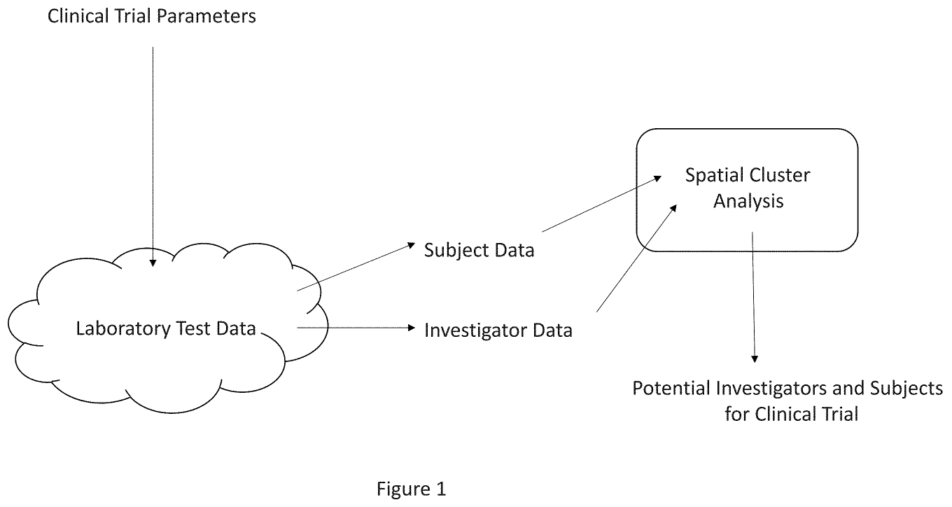

An embodiment of the present invention is depicted schematically in FIG. 1. As shown in FIG. 1, a proposed clinical trial may include a plurality of parameters. These parameters may include, but are not limited to, a patient population to be studied, treatment(s) to be investigated, endpoints, and how the trial will be conducted (e.g., randomized vs nonrandomized). A patient population may include patients who are likely to benefit from the treatment or intervention to be tested. The population may also be selected such that the results of the trial can be generalized to patients in clinical practice. Overall, the more diverse the patient population, the more generalizable the results may be to the wider patient population. A patient in a clinical trial may also be referred to as a subject and the terminology is used interchangeably herein.

In order to study a patient population of the appropriate disease state and level of diversity, investigators define criteria that determine whether or not a patient is eligible for a trial. Inclusion and exclusion criteria can include patient characteristics (e.g., age, genetic profile) as well as disease and treatment-specific characteristics including prior laboratory test results relating to the disease and/or condition. An additional parameter is the number of patients needed for the clinical trial. The clinical trial parameters may further include desired timing for enrolling patients and/or investigator sites as well as a proposed timeline for completing the clinical trial.

As shown in FIG. 1, the clinical trial parameters are used to query a laboratory test data database to determine possible subjects and investigators for a clinical trial. The database provides Subject Data relating to each possible subject including, but not limited to, the attributes set forth above and the subject's geographic location. Similarly, the database provides Investigator Data relating to each possible investigator including, but not limited to, the attributes set forth above, and the investigator's geographic location. The geographic location data for each subject and/or each investigator may comprise global positioning system coordinate data, US Postal Service ZIP code data, longitudinal and/latitudinal data and the like.

As further shown in FIG. 1, the Subject Data and Investigator Data undergo a spatial cluster analysis such as the one described above. The spatial cluster analysis outputs potential investigators and subjects for a clinical trial.

The features and advantages of embodiments of the present invention will be further understood from the following illustrative example.

### EXAMPLE

An embodiment of the present invention was utilized to select patients and investigators (clinical trial sites) for a hypothetical clinical trial.

A database maintained by the assignee of the present application, a health care diagnostics company, was used. The database included: greater than 13 billion test results; greater than 500,000 samples processed daily; over 4000 diagnostic tests; greater than 758,000 healthcare professionals provided with results; and over 142 million patients.

The hypothetical clinical trial starting parameters included the following:

150 patients with evidence of potential recurrent disease (defined as two positive

samples within 1-3 months of each other;

a 9 month timeline for enrollment in the US;

a draft site list of 120 investigators from 95 different ZIP codes (locations).

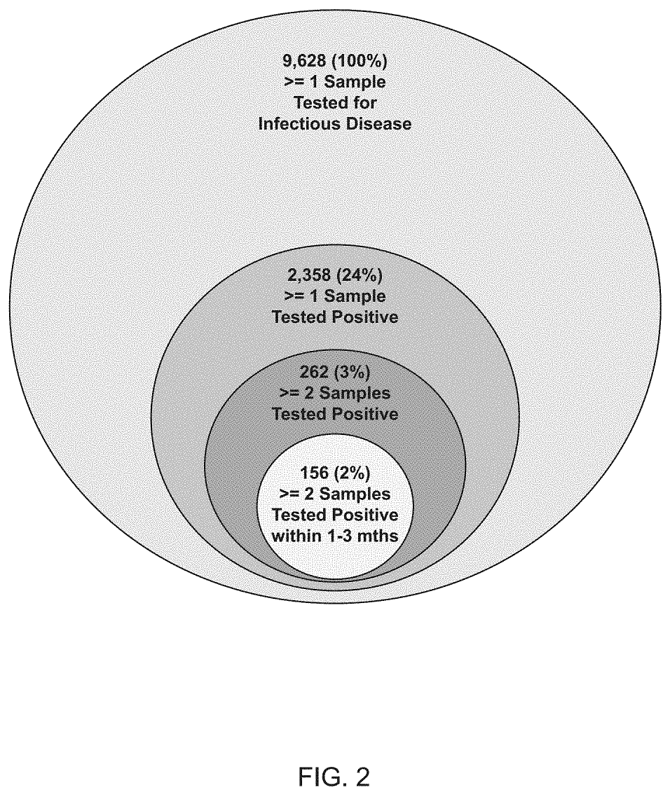

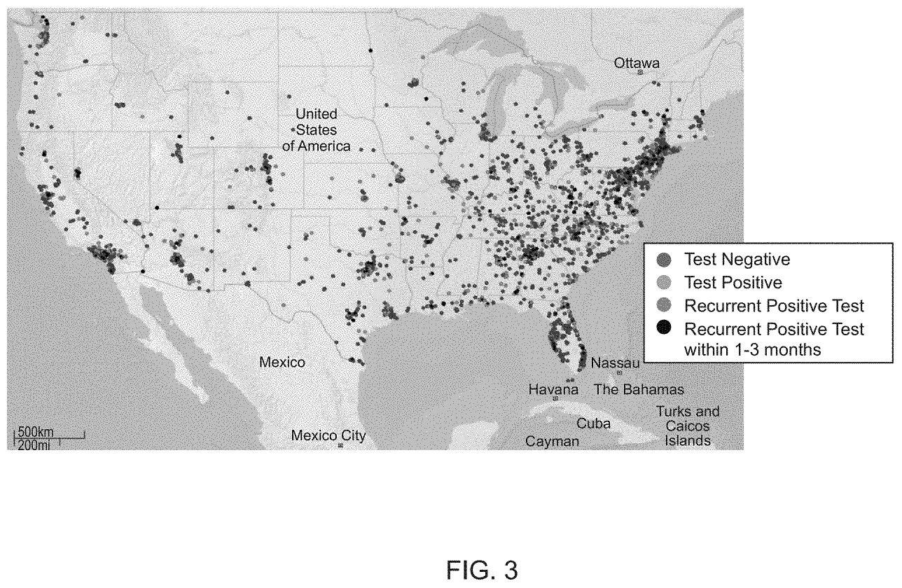

Interrogation of the previous 14 months of laboratory testing database within the database revealed that 9,628 patients from across 2,848 ZIP codes had been tested for the disease and of those 2,358 (less than 25%) had at least one sample that tested positive. Of those testing positive, 262 patients (approximately 3%) had two or more samples that tested positive. Of the 262 patients, 156 (approximately 2%) had two samples that tested positive within 1-3 months. These results are shown in FIG. 2. The geographic distribution of subjects that tested positive is shown in FIG. 3 wherein negative tests are red circles, positive tests are yellow circles, recurrent positive tests are green circles and recurrent positive tests within 1-3 months are black circles.

In view of this patient and geographic data, in the absence of the present invention, i.e. using prior art techniques, if investigators were to be enlisted at all 2,848 ZIP codes capturing all patients tested, a 13 month recruitment window would be required to provide an 80% or greater chance of randomizing 150 patients for a clinical trial. Applying a uniformity of enrollment assumption, it would take thirty times longer, i.e. over thirty-two years, using sites at only 95 ZIP codes. Realistically, in the Phase II setting, an 18-month timeline is the maximum that could be considered; under the assumption that future testing patterns mirror the historical patterns observed, coverage of ZIP codes needs to be increased in proportion to the volume of testing assuming that the number of positive tests is proportional to all testing. Thus, in the absence of the present invention, the desired timeline for enrollment cannot me met, and the logistics of the clinical trial will be difficult due to the geographic distribution of trial locations.

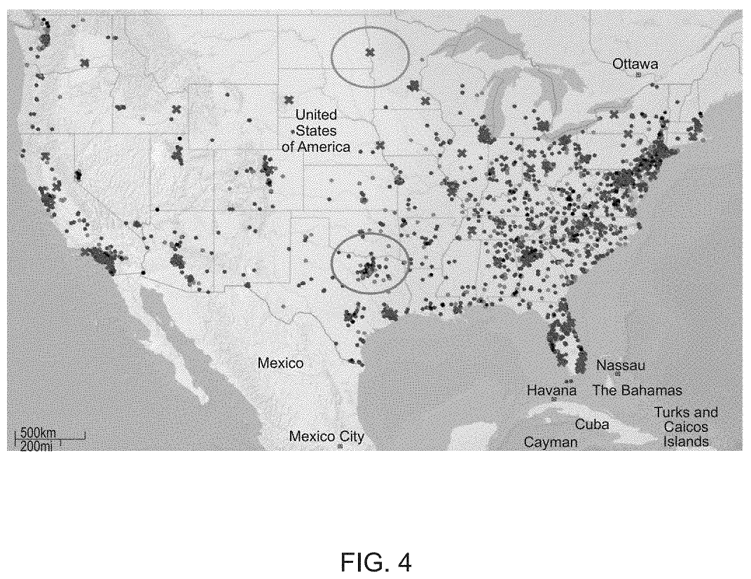

In order to meet the desired clinical trial parameters, an embodiment of the present invention was utilized. Initially, proposed investigator sites for the trial were identified. These sites are shown as blue crosses in FIG. 4, together with the patient data. As noted, there were several proposed investigator sites remote from subjects (one is circled in yellow) and several geographic locations with a large number of potential subjects without an investigator (one is circled in green).

ZIP Codes were used to determine the coordinates (latitude and longitude) of each testing center and investigator. Direct “as the crow flies” distance between each testing center and every investigator within the same US state was calculated using Haversine formula (implemented as the geodist function in SAS):

$$d = 2 r {sin}^{- 1} ( \sqrt{hav ( {lon}_{1} - {lon}_{2} ) + cos ( {lon}_{1} ) cos ( {lon}_{2} ) hav ( {lat}_{1} - {lat}_{2} )} )$$

where
hav(l)=sin⁻²(½) is the Haversine function
d—the distance between the two points
r—the radius of the sphere (specified in miles=3,959)
(lon₁,lat₁) and (lon₂,lat₂) are the longitude and latitude of each coordinate in radians
   Fifty miles was used as a general cut-off for potential referral determination using the minimum distance.

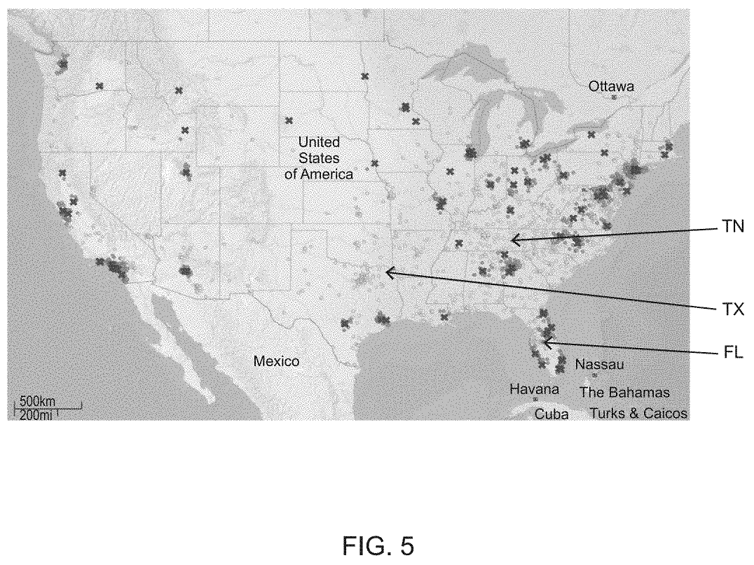

The geographic location of sites is shown in FIG. 5 wherein sites are denoted in yellow and sites less than 50 miles are denoted in blue.

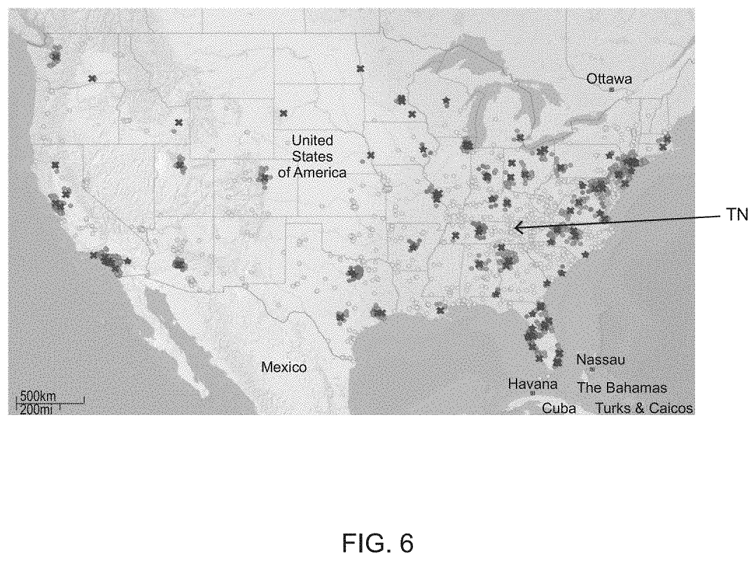

Investigators from past trials were added to the evaluation and the calculation repeated. The results are shown in FIG. 6 wherein the green stars represent sites less than 50 miles. As shown in FIG. 6, the state of Tennessee appears to have untapped potential.

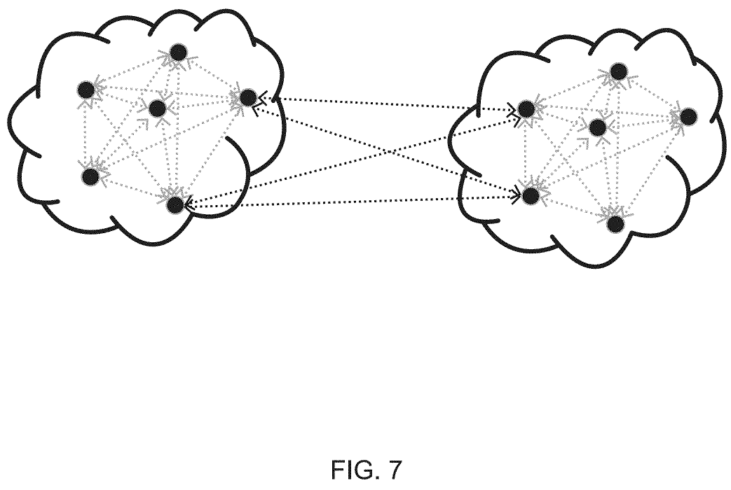

Potential new sites were identified according to the present invention through spatial clustering. The approach taken was to examine whether new investigators could be identified by calculating a distance matrix between each pair of testing centers and the use of a spatial cluster model according to the following:
ods output clusterhistory=c_ST,
proc cluster data=sqlmatz (type=distance) outtree=t_st nonorm method=complete
trim=10 r=25;
by phys_std;
copy phys_zipnd numpatd numphyd numpatposd; id phys_zipndd;
run;
   Clusters were then assessed for potential usefulness in terms of numbers of potential patients and investigator suitability. Using the Complete method, clusters were defined such that all sites within a cluster are within the distance specified from each other and the minimum distance between clusters is greater than the specified distance. Use of the trim option maintained a regular cluster shape. A schematic representation is shown in FIG. 7.

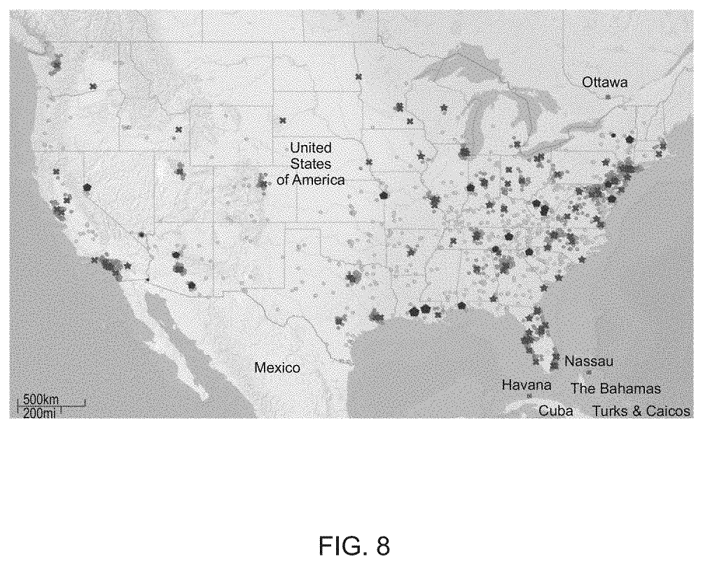

The results from the analysis are shown in FIG. 8 wherein purple pentagons denote the clusters identified from the cluster analysis with at least 30 patients. These results would be expected to allow the enrollment parameters of the clinical trial to be met.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations and/or additions to the present subject matter as would be readily apparent to one of ordinary skill in the art.

1. A method for identifying subjects for enrollment in a clinical trial comprising:
a) identifying a number of potential investigators for a clinical trial to create an investigator list;
b) determining the location of each investigator;
c) identifying a number of possible subjects for the clinical trial;
d) determining the location of each subject;
e) selecting a specified distance between a subject and an investigator; and
applying a spatial cluster analysis to determine the number of subjects within the specified distance for each investigator.

2. The method of claim 1 wherein the location of each subject comprises the location of the subject's health care provider.
